(19) United States
(12) Patent Application Publication
(10) Pub. No.: US 2024/0374868 A1
(43) Pub. Date: Nov. 14, 2024

(54) FIXING DEVICE FOR FIXING A CATHETER

(71) Applicant: B. Braun Melsungen AG, Melsungen (DE)

(72) Inventors: Iris Blum, Fuldabrueck (DE); Jacqueline Berg, Melsungen (DE)

(21) Appl. No.: 18/656,990

(22) Filed: May 7, 2024

(30) Foreign Application Priority Data
May 8, 2023 (DE) 10 2023 111 956.7

(51) Int. Cl. A61M 25/02

(52) U.S. Cl. CPC A61M 25/02; A61M 2025/0266

(57) ABSTRACT

A fixing device for fixing a catheter on skin includes a base body with a proximodistal longitudinal axis, a mediolateral transverse axis and an anteroposterior vertical axis. A posterior underside of the base body is configured for placing on the skin. A fixing element on the base body is configured for form-fit and/or force-fit fixing of a catheter hub of the catheter and by which the catheter hub is fixable relative to the base body. A guide recess is arranged distally on the base body relative to the fixing element and extends proximodistally over a guide length and has mediolaterally opposite guide walls. The guide recess is configured to receive an elongate catheter tube portion of a catheter tube of the catheter, secured distally to the catheter hub. The catheter tube portion is guidable between the guide walls over the guide length and is protected against mediolateral buckling.

### CROSS-REFERENCE TO RELATED APPLICATION(S)

This application claims priority under 35 U.S.C. § 119 to German Application No. 10 2023 111 956.7, filed on May 8, 2023, the content of which is incorporated by reference herein in its entirety.

### FIELD

The present disclosure relates to a fixing device for fixing a catheter on the skin of a patient.

### BACKGROUND

Catheters usually have a flexible catheter tube, extending between a proximal end of the tube and a distal end of the tube, and a catheter hub fastened to the proximal end of the tube.

To apply the catheter, the catheter tube is introduced into the patient's body in a known manner, for example by what is called the Seldinger technique, with the distal end of the tube to the front, through a puncture site in the skin. The catheter hub remaining outside the patient serves to fluidically connect the catheter tube to other fluid-carrying components, for example a catheter supply line or the like. Moreover, the catheter tube is usually not inserted over its entire length into the patient, such that a catheter tube portion which distally adjoins the catheter hub, and which can also be referred to as the free catheter length, remains outside the patient.

It is customary to fix catheters on the patient's skin after they have been applied. This is intended to avoid inadvertent slipping of the catheter or even undesired withdrawal of the catheter tube from the puncture site and the associated harm to the patient. Various fixing devices are known for this purpose.

For example, fixing devices in the form of adhesive pads are known, which can be glued to the skin and are configured for fastening a catheter filter of the catheter that is to be fixed. Neither the catheter hub nor the catheter tube of the catheter is fixed directly. Such fixing devices are currently marketed by, among others, the company B. Braun Melsungen under the registered trademark PERIFIX® PINPAD or by the company Pajunk under the name FIXOLONG™.

Furthermore, fixing devices are known which can likewise be glued to the skin and are configured for fastening the catheter hub of the catheter that is to be fixed. These fixing devices also do not provide fixing of the catheter tube distally adjoining the catheter hub. Such fixing devices are currently marketed by, among others, the company Pajunk under the name FIXOCATH™ or by the company Bard Medical under the registered trademark STATLOCK®.

### SUMMARY

The object of the present disclosure is to make available a fixing device of the type mentioned above, which enables improved patient safety.

The fixing device according to the present disclosure has a base body, a fixing element and a guide recess. The base body has a proximodistal longitudinal axis, a mediolateral transverse axis and an anteroposterior vertical axis, and also a posterior underside. The posterior underside is configured for at least indirect application to the patient's skin. The fixing element is arranged on the base body and is configured for the form-fit and/or force-fit fixing of a catheter hub of the catheter. By means of the fixing element, the catheter hub is fixable and/or fastenable relative to the base body. The guide recess is arranged distally on the base body relative to the fixing element and extends proximodistally over a guide length. The guide recess has mediolaterally opposite guide walls. The guide recess is configured to receive an elongate catheter tube portion, of a catheter tube of the catheter, secured distally to the catheter hub. The catheter tube portion is guidable between the guide walls over the guide length and is thus protected against mediolateral buckling. The solution according to the present disclosure primarily counteracts said lateral buckling or even folding of the catheter tube portion and the associated harm to the patient. This enables improved patient safety. In addition, improved patient comfort can be achieved. The guide recess extending along the longitudinal axis, with the guide walls opposite each other along the transverse axis, is present for this purpose. In a fixed state, the catheter tube portion distally adjoining the catheter hub is arranged between said guide walls. In this way, the catheter tube portion is held at least mediolaterally and/or laterally, in particular with limited mobility, between the guide walls and in this way is protected against buckling or even folding. The fixing element serves to releasably fasten the catheter hub to the base body. In the fixed state of the catheter, the catheter hub is fixed, by means of the fixing element, relative to the base body at least anteriorly, i.e. against lifting upward. Alternatively or additionally, the catheter hub is fixed mediolaterally. For this purpose, the fixing element interacts in a form-fitting and/or force-fitting manner with the catheter hub. The fixing element is designed differently in different embodiments, for example as a clamping, latching and/or plug-in element. The base body can basically have any suitable shape. The base body has the longitudinal axis, the transverse axis and the vertical axis. These axes are of course to be understood as geometric axes and form a Cartesian axis system. The posterior underside of the base body faces the patient's skin during the use of the fixing device. For the purposes of fixing, the posterior underside is placed directly or indirectly on the skin. The fixing can be carried out using further aids, such as a plaster, a bandage or the like. Alternatively or in addition, direct or indirect gluing of the base body to the skin is provided. It will be appreciated that the catheter to be fixed is not part of the fixing device. However, the present disclosure also relates to an arrangement with a fixing device according to the present disclosure and with a catheter fixed or to be fixed according to the preceding description.

The position and direction information used in this description, such as proximal, distal, proximodistal, medial, lateral, mediolateral, anterior, posterior and anteroposterior, serve to simplify the description and refer to a state of use of the fixing device in which the latter may be arranged on the patient's skin together with the catheter. “Distal” means in the direction of the puncture site. “Proximal” means away from the puncture site. “Posterior” means in the direction of the patient's skin located below the fixing device. “Anterior” means facing away from the skin. “Medial” and “lateral” refer to opposite directions parallel to the skin located below the fixing device. Alternatively or additionally, the indications “proximal” and “distal” indicate mutually opposite directions along the longitudinal axis. The same applies analogously to the indications “medial” and “lateral” and to the indications “anterior” and “posterior” in relation to the vertical axis.

In one embodiment of the present disclosure, the guide walls and/or the base body have/has length markings spaced proximodistally apart from one another and distributed over the guide length, each representing a defined length portion of the guide length, wherein the base body is able to be severed at least in the region of the length markings. This embodiment of the present disclosure is based on the consideration that the catheter tube portion, i.e. the free catheter length, can be of different lengths. By virtue of the available length markings and the fact that the base body can be severed, the guide length, and thus the length of the guide recess and of the guide walls, can be easily adapted, in particular by shortening, to the proximodistal length of the catheter tube portion that is to be guided. The length markings are preferably distributed equidistantly. In different embodiments, the length markings are designed differently, for example as countersinks, bulges, numbers, letters, characters or the like. The ability of the base body to be severed can be achieved in particular by a suitable geometric design and/or choice of material. Preferably, the length markings form, as it were, predetermined separation points for the defined severing of the base body. The predetermined separation points each form a mechanical weakening of the base body.

In a further embodiment of the present disclosure, the guide walls and/or the base body have/has predetermined separation points spaced proximodistally apart from one another and distributed over the guide length, each forming a mechanical weakening and being able to be severed in a defined manner. This embodiment of the present disclosure is also based on the consideration that the free catheter length can vary depending on the application. By means of the predetermined separation points, the guide length can be easily adapted, in particular by shortening it, to the length of the catheter tube portion that is to be guided. For this purpose, the base body is severed in a defined manner in the region of the predetermined separation points, for example by means of the base body being torn, cut or otherwise split in the region of one of the predetermined separation points. Each of the predetermined separation points forms a mechanical weakening. For this purpose, the predetermined separation points can each form a local reduction in wall thickness, a notch or the like. In addition, it is conceivable and possible for predetermined separation points to be provided in each case in the form of a perforation along which the base body can be separated. Preferably, each of the predetermined separation points forms a length marking, which in each case represents a defined length portion of the guide length.

In a further embodiment of the present disclosure, the guide walls are each formed by proximodistally spaced apart web elements with intermediate gaps. The web elements form two web rows lying opposite each other mediolaterally, i.e. along the transverse axis, with one of the web rows in each case being assigned to one of the two guide walls. The web elements and/or the intermediate gaps are preferably spaced apart equidistantly from one another. Preferably, the web elements and/or gaps are each arranged on an anterior top of the base body. The web elements with the intermediate gaps permit in particular an improved view of the catheter tube portion received in the guide recess.

In a further embodiment of the present disclosure, the gaps arranged between the web elements each function as one of the length markings and/or one of the predetermined separation points. As regards the properties and function of the length markings and/or of the predetermined separation points, the statements concerning the previous embodiments apply analogously, and reference is expressly made to these in order to avoid repetition.

In a further embodiment of the present disclosure, the proximodistal guide length measures between 10 mm and 60 mm, preferably between 40 mm and 50 mm. It has been found that the above value ranges for the guide length are particularly consistent with requirements for effectively counteracting a lateral buckling of the catheter tube portion. If the guide walls and/or the base body are provided with length markings and/or predetermined breaking points, the guide length can be adapted in the manner described above. For example, the guide length, in this case of 60 mm, can be shortened to 10 mm, and intermediate values are of course also possible according to the available length markings and/or predetermined separation points.

In a further embodiment of the present disclosure, the fixing element and the guide recess are each arranged on an anterior top of the base body. The top lies opposite the underside along the vertical axis. When the fixing device is in use, the top is facing away from the patient's skin. The arrangement of the fixing element and of the guide recess on the top of the base body improves the visibility of the fixed portions of the catheter. This makes it possible to detect incorrect fixing in an improved way.

In a further embodiment of the present disclosure, the fixing element and the guide recess are each open anteriorly, as a result of which the catheter hub and the catheter tube portion are each insertable in the posterior direction into the fixing device. By virtue of the fixing element being open at the top, the catheter hub can be inserted into the fixing element in a simple and ergonomic manner along the vertical axis in the posterior direction, i.e. from the top downward. The same applies analogously to the insertion of the catheter tube portion in the guide recess. In addition, this embodiment allows an advantageous distribution of the forces that act on the patient's skin upon insertion of the catheter hub and of the catheter tube portion.

In a further embodiment of the present disclosure, the fixing element and the guide recess are each open posteriorly, as a result of which the fixing device can mounted in the posterior direction onto the catheter hub and the catheter tube portion. By virtue of the guide recess being open downward along the vertical axis, it can be mounted onto the catheter tube portion in the posterior direction, i.e. from the top downward along the vertical axis. The same applies analogously to the fixing element in relation to the catheter hub. This is a particularly preferred embodiment of the present disclosure. In particular, in contrast to the preceding embodiment, the posteriorly open design of the guide recess and of the fixing element allows fixing to be carried out while largely or even completely avoiding manipulation of the already applied catheter. In simple terms, the fixing device in this embodiment of the present disclosure can be applied from the top downward onto the already applied catheter. By contrast, in the previous embodiment, the already applied catheter first has to be manually lifted from the skin. Thereafter, with the catheter lifted, the fixing device can be pushed under the latter, and the catheter can then be inserted posteriorly into the fixing device.

In a further embodiment of the present disclosure, the underside has a recess which extends proximodistally over the entire length of the base body and which divides the base body into a medial body half and a lateral body half and is formed in part by the guide recess, wherein the two body halves are connected to each other, preferably elastically movably, at least by means of the fixing element. By this embodiment of the present disclosure, the fixing device can be mounted in a particularly simple and ergonomic manner in the posterior direction onto the already applied catheter. The recess extends along the longitudinal axis and preferably continuously along the vertical axis between the posterior underside and the anterior top of the base body. This preferably also applies to the guide recess, which forms part of said recess. The medial body half and the lateral body half lic opposite each other along the transverse axis. The elongate recess preferably forms a line of symmetry. Preferably, the base body is mirror symmetrical with respect to the elongate recess. The medial body half and the lateral body half are connected to each other at least by means of the fixing element. It will be appreciated that further transverse connections may be present between the two halves of the body. In a preferred embodiment, a flexure bearing connecting the body halves is present, which is preferably formed by the fixing element, such that the two body halves are connected to each other by means of the flexure bearing so as to be movable to a limited extent in a hinged manner, preferably elastically. In this case, the relative mobility between the body halves takes place about an imaginary (virtual) articulation axis oriented parallel to the elongate recess. In such a design, the two body halves can be pivoted away from each other for simplified application of the fixing device to the already applied catheter. The pivoting movement of the two body halves widens the guide recess in the mediolateral direction. After application, an opposite pivoting movement takes place, such that the catheter hub is fixed in a form-fitting and/or force-fitting manner by means of the fixing element and the catheter tube portion is guided in the once again narrowed guide recess.

In a further embodiment of the present disclosure, a stop element is present which is arranged proximally on the base body relative to the fixing element and by means of which at least a proximal mobility of the catheter hub and/or of a fluid-carrying component of the catheter attached to the catheter hub can be positively limited. The stop element functions as a strain relief for proximally directed tensile forces. In this way, an unintentional withdrawal of the catheter tube from the puncture site can be counteracted in a further improved manner. To limit the proximal mobility of the catheter hub, the stop element preferably interacts directly with the catheter hub positively. Alternatively or in addition, the stop element acts directly positively on said component, such that the mobility of the catheter hub is indirectly positively limited.

In a further embodiment of the present disclosure, the base body is made of a dimensionally stable plastics material. In particular, sufficient mechanical strength is achieved in this way. Despite being produced from the dimensionally stable plastics material, the base body can be designed to be dimensionally flexible by means of a corresponding design, in particular by corresponding dimensioning of its wall thicknesses. In a further embodiment of the present disclosure, the base body, relative to its proximodistal and its mediolateral extent, is thin along the vertical axis and/or is designed in the form of a proximodistally and mediolaterally planar plate. By virtue of a thin and/or plate-shaped design of the base body, the latter lies comparatively close to the patient's skin during use. This improves patient comfort. In addition, the thin and/or plate-shaped design avoids a situation where the base body becomes caught on the patient's clothing or the like and is thereby inadvertently moved. In addition, this embodiment allows a particularly advantageous distribution of the forces that act on the patient's skin upon insertion of the catheter hub and of the catheter tube portion. This is especially the case when the fixing element and the guide recess are each open anteriorly, as a result of which the catheter hub and the catheter tube portion are each insertable in the posterior direction into the fixing device.

In a further embodiment of the present disclosure, a dimensionally flexible cushion body is present, on the anterior top of which the base body is firmly applied, and of which the posterior underside is configured for placing on the skin, in particular wherein a contact surface formed by the underside of the cushion body is larger by a factor of 1.2 to 3.0, preferably 1.5 to 2.5, than the underside of the base body. The dimensionally flexible cushion body permits a further improvement in patient comfort. For this purpose, the cushion body is arranged, in relation to the vertical axis, between the patient's skin and the base body and is dimensionally flexible. The improved patient comfort is achieved by the dimensional flexibility of the cushion body. In a preferred embodiment, the cushion body is made of a foamed plastics material and/or is elastic. In this embodiment of the present disclosure, the underside of the base body, during the use of the fixing device, is applied only indirectly to the skin. The actual contact surface of the fixing device is formed by the underside of the cushion body. This surface is preferably larger than the underside of the base body by a factor within the above-mentioned value ranges. This results in a reduced pressure load on the skin. This helps to further improve patient comfort.

In a further embodiment of the present disclosure, an adhesive coating assigned to the underside of the base body is present and is configured for releasably gluing the fixing device to the skin. The adhesive coating can be arranged directly on the underside of the base body. If a cushion body is present, the adhesive coating is preferably arranged on the underside thereof. In a delivery state of the fixing device, the adhesive coating is preferably covered by a peel-off film. After the film has been peeled off, the fixing device can be applied to the patient's skin, with the adhesive coating to the front, and in this way affixed in a releasable manner. This permits particularly simple fixing without the use of other aids.

### BRIEF DESCRIPTION OF THE DRAWINGS

Further advantages and features of the present disclosure will become clear from the following description of preferred exemplary embodiments of the present disclosure, which are shown in the drawings.

FIG. 1 shows a schematic plan view of an embodiment of a fixing device according to the present disclosure for fixing a catheter,

FIG. 2 shows the fixing device according to FIG. 1 in a schematic side view,

FIG. 3 shows a schematic plan view of a catheter that can be fixed by means of the fixing device according to FIGS. 1 and 2,

FIG. 4 shows a schematic plan view of an example of a use situation, in which the catheter according to FIG. 3 is fixed on a patient's skin by means of the fixing device according to FIGS. 1 and 2, and

FIG. 5 shows a schematic plan view of a further embodiment of a fixing device according to the present disclosure.

### DETAILED DESCRIPTION

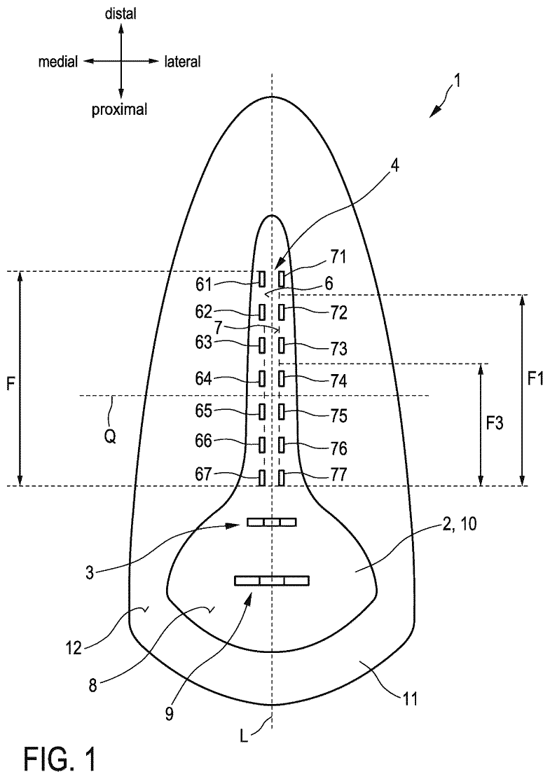

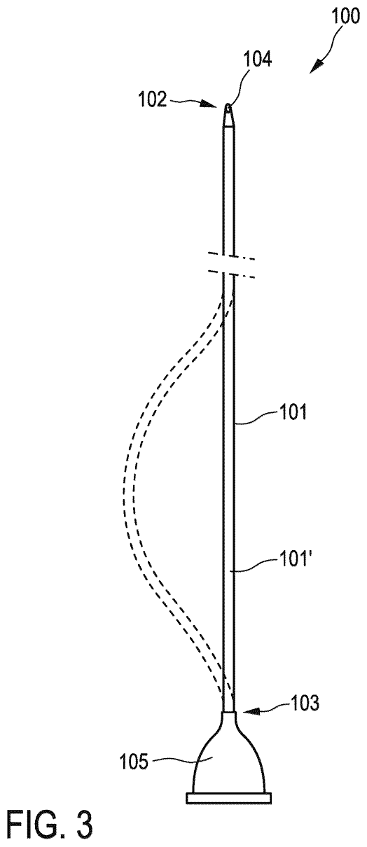

According to FIGS. 1, 2 and 4, a fixing device 1 is provided for fixing a catheter 100 (FIG. 3) on the skin S of a patient. The fixing device 1 is suitable for fixing conventional catheters of the kind used, for example, in infusion therapy, transfusion therapy and/or pain therapy.

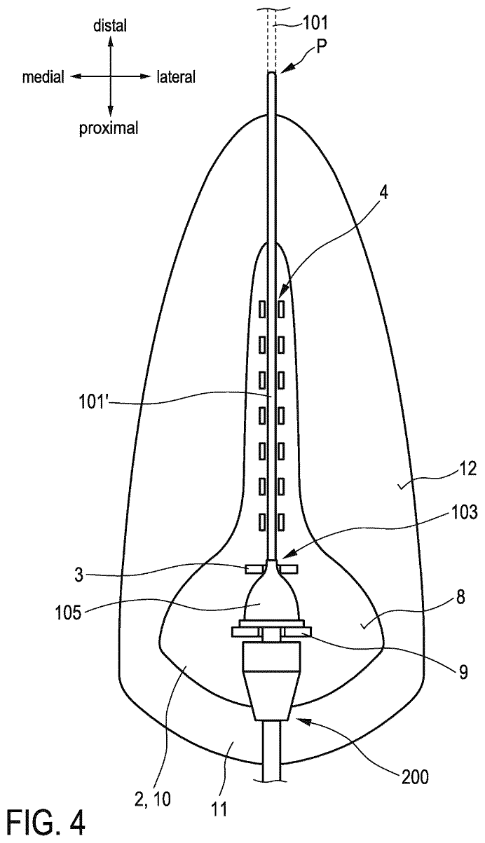

The catheter 100 shown as an example in FIG. 3 has a structure known to a person skilled in the art. The catheter 100 has a catheter tube 101 which extends between a distal catheter tube end 102 and a proximal catheter tube end 103. The catheter tube 101 has a lumen (not shown in detail) which, in the region of the distal catheter tube end 102, opens into a catheter outlet 104. By way of the catheter outlet 104, medical liquids can be dispensed from the lumen of the catheter tube 101, or body fluids or other liquids can be aspirated into the lumen. The catheter 100 also has a catheter hub 105. The catheter hub 105 is firmly connected to the proximal tube end 103. The catheter hub 105 serves primarily to fluidically connect the catheter tube 101 to further fluid-carrying components, for example a catheter supply line 200 (see FIG. 4) or the like. The catheter tube 101 is made of a flexible plastics material. The resulting deformability of the catheter tube 101 is illustrated in FIG. 3 by the dashed lines. It will be appreciated that the design of the catheter 100 shown in FIG. 3 is greatly simplified and purely illustrative.

The fixing device 1 has a base body 2, a fixing element 3 and a guide recess 4.

The base body 2 has a longitudinal axis L, a transverse axis Q and a vertical axis H. These axes L, Q, H form a Cartesian axis system and are orthogonal to one another. The longitudinal axis L is oriented proximodistally. The transverse axis Q is oriented mediolaterally. The vertical axis H is oriented anteroposteriorly.

As regards the use situation of the fixing device 1 shown in FIG. 4, said position and orientation information can be understood as follows: “Distal” means in the direction of the distal catheter tube end 102 and/or the catheter outlet 104, “proximal” means in the direction away from the distal catheter tube end 102 and/or in the direction of the proximal catheter tube end 103, “posterior” means in the direction of the patient's skin S, “anterior” means in the direction away from the skin S, and “medial” and “lateral” refer to opposite directions parallel to the patient's skin S. It will be appreciated that said position and direction information is not strictly necessary for a full understanding of the underlying present disclosure, but merely serves the purposes of a simplified description.

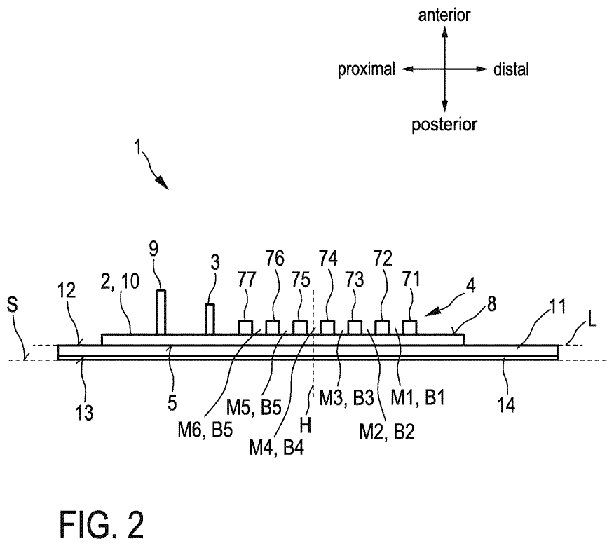

The base body 2 also has an underside 5, which is configured to be placed at least indirectly on the skin S (see FIG. 2).

The fixing element 3 is arranged on the base body 2 and serves for the form-fit and/or force-fit fixation of the catheter hub 105 of the catheter 100 that is to be fixed. The fixing element 3 is configured accordingly and, in the embodiment shown, is designed in the manner of a latching element. Alternatively, the fixing element can be designed as a plug-in and/or clamping element. Consequently, for the purpose of fastening it to the base body, the catheter hub 105 can be latched, clamped and/or plugged onto the fixing element 3. Details of the design of the fixing element 3 in this respect are not essential as regards the present disclosure. It is important only that the fixing element 3 is configured for the form-fit and/or force-fit fixation of the catheter hub 105. The fixing element 3 interacts for this purpose directly with the catheter hub 105 and has, for example, a latching, clamping and/or plug-in geometry, which is designed to complement a geometry of the catheter hub 105. The fixing element 3 is configured for releasable fixation, such that the previously fixed catheter hub can at any time be released again from the base body 2 if necessary.

The guide recess 4 is arranged on the base body 2 and extends along the longitudinal axis L over a guide length F. In the present case, the guide recess 4 is parallel to the longitudinal axis L. The fixing element 3 is arranged at a proximal end (not shown in detail) of the guide recess 4. The guide recess 4 has two guide walls 6, 7 lying opposite each other along the transverse axis Q. In the present case, the guide walls 6, 7 can also be referred to as medial guide wall 6 and lateral guide wall 7, or vice versa. The guide recess 4 is configured to receive a catheter tube portion 101′ of the catheter tube 101. The catheter tube portion 101′ to be received extends distally starting from the catheter hub 105 and/or the proximal catheter tube end 103. In other words, the catheter tube portion 101′ that can be received by means of the guide recess 4 is directly adjacent to the catheter hub 105. To put it another way, the catheter tube portion 101′ to be received is a proximal portion of the catheter tube 101. The catheter tube portion 101′ is able to be guided between the guide walls 6, 7 of the guide recess 4.

In the use situation of the fixing device 1 shown in FIG. 4, the catheter 100 is applied in a manner known to a person skilled in the art. For applying the catheter 100, the catheter tube 101 is inserted, with the distal catheter tube end 102 to the front, through a puncture site P into the skin S and the underlying body tissue of the patient. This is done, for example, by means of what is called the Seldinger technique. The catheter hub 105 remains outside the patient and spaced proximally from the puncture site P. Moreover, the catheter tube 101 is not fully inserted through the puncture site P, such that at least the catheter tube portion 101′ directly distal to the catheter hub 105 remains outside the patient. The catheter tube portion 101′ is often also referred to as the free catheter length. Depending on how far the catheter tube 101 is advanced distally through the puncture site P, the length of the catheter tube portion 101′ changes.

In the illustrated use situation of the fixing device 1, the base body 2, with its underside 5 leading, is applied (at least indirectly) to the skin S and is releasably connected to the latter in a manner to be described in more detail. The catheter hub 105 is fastened releasably to the base body 2 by means of the fixing element 3 and, in the present case, is for this purpose latched onto the fixing element 3. In the embodiment shown, the catheter hub 105 is held in a fixed state, at least along the transverse axis Q and along the vertical axis H, relative to the base body 2 in a form-fitting and/or force-fitting manner. In addition, the catheter hub 105 in the present case is held along the longitudinal axis L, at least in the distal direction, in a form-fitting and/or force-fitting manner by means of the fixing element 3.

Furthermore, the catheter tube portion 101′ is received in the guide recess 4, is guided between the guide walls 6, 7 thereof and, as a result, is protected at least against mediolateral buckling. In the embodiment shown, an unspecified transverse distance between the guide walls 6, 7 is dimensioned in such a way that the catheter tube portion 101 is held with limited mobility in its radial direction, i.e. along the transverse axis Q, between the guide walls 6, 7. Consequently, the catheter tube portion 101 is not clamped or elastically deformed by means of the guide walls 6, 7. In an embodiment not shown in the figures, the transverse distance between the guide walls can be dimensioned in such a way that the guide recess exerts a clamping action on the catheter tube portion.

In the embodiment shown, the guide length of the guide recess 4 and thus also of the guide walls 6, 7 measures 50 mm. In embodiments not shown in the figures, the guide length measures between 10 mm and 60 mm, preferably between 40 mm and 50 mm.

In the embodiment shown, the guide walls 6, 7 do not extend continuously and/or in an uninterrupted manner over the entire guide length F. Instead, the guide walls 6, 7 are each formed by web elements 61 to 67 and 71 to 77 spaced apart proximodistally from one another.

The web elements 61 to 67 are assigned to the medial guide wall 6 and can therefore also be referred to as medial web elements.

The web elements 71 to 77 are assigned to the lateral guide wall 7 and can therefore also be referred to as lateral web elements.

The web elements 61 to 67 and 71 to 77 are spaced proximodistally and equidistantly from one another over the guide length F.

In the embodiment shown, the webs are each 5 mm long. In embodiments not shown in the figures, the webs are each between 5 mm and 20 mm long. The gaps (without reference signs) between two proximodistally adjacent web elements are dimensioned accordingly. The guide walls 6, 7 extend parallel to each other. The same therefore also applies to the mutually opposite medial web elements 61 to 67 and lateral web elements 71 to 77.

In the embodiment shown, the gaps arranged between the web elements each function as a length marking M1 to M6 and/or as a predetermined separation point B1 to B6.

The length markings M1 to M6 formed by the gaps each represent a defined length portion of the guide length F. The length markings M1, M2 can also be referred to as first length marking M1 and second length marking M2. The same applies analogously to the designation of the further length markings M3 to M6. The first length marking M1 represents a first guide sub-length F1. The third length marking M3 represents a third guide sub-length F3. The same applies analogously to the further length markings. In other words, the length markings M1 to M6 form a defined division of length for the guide recess 4 and its guide walls 6, 7.

As has been mentioned above, the gaps (without reference signs) alternatively or additionally form the predetermined separation points B1 to B6. The predetermined separation points B1 to B6 each form a local mechanical weakening of the base body 2. These local mechanical weakenings allow defined severing of the base body 2 at said predetermined separation points B1 to B6. In the present case, the mechanical weakening is provided by a reduction in the material thickness of the base body 2 in the region of the gaps. At the places with reduced material thickness, i.e. at the gaps and/or predetermined separation points B1 to B6, the base body 2 can therefore be severed quite easily. For example, the base body 2 can be severed manually or by means of a cutting tool or the like. Tearing off is also conceivable and possible. Alternatively or additionally, the length markings M1 to M6 and/or the predetermined separation points B1 to B6 can be formed in each case by a perforation of the base body 2, the perforation extending parallel to the transverse axis Q. The predetermined separation points B1, B2 can also be referred to as first predetermined separation point B1 and second predetermined separation point B2. The same applies analogously to the designation of the further predetermined separation points B3 to B6.

The length markings M1 to M6 and/or the predetermined separation points B1 to B6 allow a simplified adaptation, in particular shortening, of the guide length to the actual free length of the catheter tube 101. If the catheter tube 101 is to be advanced further distally through the puncture site P in comparison to the use situation shown in FIG. 4, the guide length can be easily shortened by severing the base body 2 at a location suitable for this purpose. For example, the guide length F can be shortened to the guide sub-length F1 by severing the base body 2 at the gap (length marking M1, predetermined separation point B1) formed between the web elements 61 and 62 and 71 and 72. If the catheter tube 101 is to be distally advanced still further, the base body 2 can be shortened, for example, to the third guide sub-length F3. For this purpose, the base body 2 is severed in a defined manner at the gap (third length marking M3, third predetermined separation point B3) located between the guide webs 63, 64 and 73, 74.

In the embodiment shown in FIGS. 1, 2 and 4, the guide recess 4 and the fixing element 3 are each open in the anterior direction. Through the respective anterior opening, the catheter hub 105 and the catheter tube portion 101′ can be inserted in each case in the posterior direction into the fixing device 1. More specifically: In the present case, the catheter hub 105 is latched onto the fixing element 3 from above, and the catheter tube portion 101′ is inserted into the guide recess 4 from above. For fixing purposes, the already applied catheter 100 is raised anteriorly and away from the skin S. The fixing device 1 is pushed preferably laterally under the raised catheter 100 and applied to the skin S. Thereafter, the raised catheter 100 can be lowered and inserted into the fixing device 1 in the manner described above.

In the embodiment shown in FIGS. 1, 2 and 4, the fixing element 3 and the guide recess 4 are each arranged on an anterior top 8 of the base body 2. In other words, the guide walls 6, 7 and the fixing element 3 each protrude anteriorly from the base body and/or its top 8.

In the present case, the fixing device 1 also has a stop element 9. The stop element 9 is arranged on the base body 2 and is spaced proximally from the fixing element 3 and/or the guide recess 4. The stop element 9 is configured to positively limit a proximal mobility of the catheter hub 105. In other words, the stop element 9 functions as a direct or indirect stop for the catheter hub 105. In this way, the stop element 9 acts as a strain relief, which counteracts a proximal pulling-out of the catheter tube 100 from the puncture site P.

In the embodiment shown, the stop element 9 interacts directly with a proximal end (not shown in detail) of the catheter hub 105. To limit the mobility, the stop element 9 contacts said proximal end of the catheter hub 105. In an embodiment not shown in the figures, the stop element instead interacts with the catheter supply line 200 (FIG. 4). The stop element 9 is in turn arranged on the top 8 of the base body 2 and protrudes anteriorly from same.

In the embodiment shown, the base body 2 is designed in the form of a plate 10 extending in planar fashion in the proximodistal and mediolateral directions. The plate 10 can also be referred to as base plate. In the present case, the base body 2 is thus relatively thin along the vertical axis H and has a low material thickness in comparison to its planar extent.

In the present case, the base body 2 is elongate along the longitudinal axis L and comparatively narrow along the transverse axis Q. In addition, there is a symmetry with respect to the longitudinal axis L and thus also to the guide recess 4. It will be appreciated that the design of the entire fixing device 1, in particular of the base body 2, shown in the figures is to be regarded as purely illustrative.

In addition, the fixing device 1 has a dimensionally flexible cushion body 11 in the present case. The cushion body 11 has an anterior top 12 and a posterior underside 13. The cushion body 11 is arranged, with respect to the vertical axis H, between the base body 2 and the skin S and serves as a soft elastic support for improving patient comfort. The base body 2 is firmly connected to the top 12 of the cushion body 11. The underside 13 of the latter is configured for placing on the skin S. Therefore, in the present case, the underside 5 of the base body 2 lies only indirectly on the skin S. In the embodiment shown, the cushion body 11 is expanded from an elastic plastics material. The cushion body 11 can therefore also be referred to as a foam body or foam support. The underside 13 of the cushion body 11 is larger than the underside 5 of the base body 2. As a result, a reduced surface load on the skin S is achieved. This reduces pressure points or other irritations on the skin S. In the embodiment shown, a contact surface (without reference sign) formed by the underside 13 of the cushion body 11 is larger, by a factor of 2.5, than the surface area of the underside 5 of the base body 2. In embodiments not shown in the figures, this factor is between 1.2 and 3.0.

For fastening purposes, the base body 2 in the present case is glued to the cushion body 11. Basically, however, other joining techniques for fastening the base body 2 to the cushion body 11 are conceivable and possible.

In the embodiment shown, the underside 13 of the cushion body 11 also has an adhesive coating 14. The adhesive coating 14 is configured for releasably gluing the fixing device 1 to the skin S. The adhesive coating 14 can be formed from a glue suitable for this purpose. In an embodiment not shown in the figures, the fixing device has no cushion body, and therefore the underside of the base body 2 lies directly on the skin. In this case, the underside of the base body 2 can be provided with an adhesive coating. In a delivery state of the fixing device 1, the adhesive coating 14 is covered by a film (not shown in detail). The film can be peeled off.

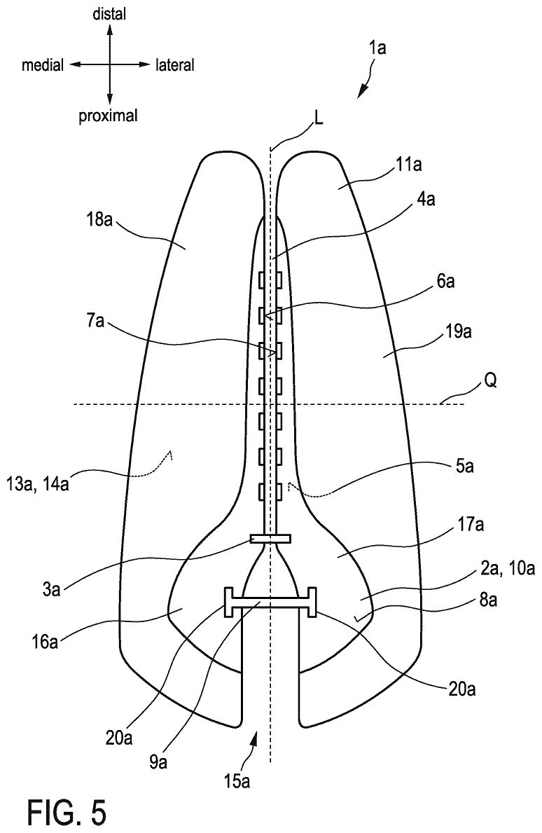

FIG. 5 shows a further embodiment of a fixing device 1a. In terms of its structure and function, the fixing device 1a according to FIG. 5 is substantially identical to the fixing device 1 from FIGS. 1, 2 and 4. To avoid repetition, only the main differences of the fixing device 1a according to FIG. 5 from the fixing device 1 according to FIGS. 1, 2 and 4 are discussed below. Otherwise, what has been disclosed regarding the fixing device 1 also applies to the fixing device 1a, and therefore reference is expressly made to what has been said regarding the embodiment according to FIGS. 1, 2 and 4.

The fixing device 1a mainly differs from the fixing device 1 in that the guide recess 4a and the fixing element 3a are open posteriorly, such that the fixing device 1a can be applied along the vertical axis H from the top downward onto the already applied catheter 100.

In the present case, the fixing device 1a has a proximodistally extending recess 15a, which divides the base body 2a into a medial body half 16a and a lateral body half 17a. The recess 15a in the present case extends over the entire length of the base body 2a. In this case, the guide recess 4a forms a portion of the recess 15a.

The fixing device 1a is in turn provided with an optional cushion body 11a. The latter is also divided into two body halves 18a, 19a by means of the recess 15a. The recess 15a in the present case extends over the entire length of the cushion body 11a.

The medial body half 16a and the lateral body half 17a of the cushion body 11a are symmetrical with respect to the longitudinal axis L and/or to the recess 15a. The same applies analogously to the two body halves 18a, 19a of the cushion body 11a.

The two body halves 16a, 17a of the base body 2a are in the present case connected to each other by means of the fixing element 3a. The fixing element 3a bridges the guide recess 15a along the transverse axis Q and is connected at one end to the medial body half 16a and at the other end to the lateral body half 17a.

The fixing device 1a in turn has an optional stop element 9a. In the present case, the stop element 9a bridges the recess 15a and forms a further mechanical connection between the two body halves 16a, 17a.

In the present case, the stop element 9a has grip portions 20a, which serve for simplified handling of the fixing device 1a. The grip portions 20a are arranged along the transverse axis Q on the outside of the stop element 9a and can be gripped, for example, between the thumb and the index finger of a hand.

In the embodiment shown, the fixing element 3a and the stop element 9a function in the manner of an articulated connection between the two body halves 16a, 17a. In other words, the fixing element 3a and/or the stop element 9a form/forms a flexure bearing, which allows a pivoting movement, directed about the longitudinal axis L, between the two body halves 16a, 17a.

By squeezing the grip portions 20a between thumb and index finger, the body halves 16a, 17a are pivoted in the present case about the longitudinal axis L and anteriorly toward each other. In this way, the guide walls 6a, 7a are moved away from each other with respect to the transverse axis Q, which leads to a widening of the guide recess 4a. In this way, the fixing device 1a can be applied in a simplified manner to the already applied catheter 100. By virtue of a suitable choice of material, the hinge properties are in the present case elastic such that, after the fingers have been released from the grip portions 20a, the body halves 16a, 17a pivot automatically toward each other about the longitudinal axis L.

In an embodiment not shown in the figures, the fixing element and the guide recess are open on both sides with respect to the vertical axis. With such a design, the fixing device can be applied posteriorly to the already applied catheter or, conversely, the catheter can be raised and lowered posteriorly for insertion into the fixing device.

1. A fixing device for fixing a catheter on skin, the fixing device comprising:
a base body with a proximodistal longitudinal axis, a mediolateral transverse axis and an anteroposterior vertical axis, and with a posterior underside configured for placing on the skin;
a fixing element arranged on the base body and configured for form-fit and/or force-fit fixing of a catheter hub of the catheter and by which the catheter hub is fixable relative to the base body; and
a guide recess arranged distally on the base body relative to the fixing element and which extends proximodistally over a guide length and has guide walls that are mediolaterally opposite to one another,
the guide recess being configured to receive a catheter tube portion of a catheter tube of the catheter, secured distally to the catheter hub, and
the catheter tube portion being guidable between the guide walls over the guide length and is thus protected against mediolateral buckling.

2. The fixing device according to claim 1, wherein the guide walls and/or the base body have/has length markings spaced proximodistally apart from one another and distributed over the guide length, each representing a defined length portion of the guide length, wherein the base body is able to be severed at least in a region of the length markings.

3. The fixing device according to claim 1, wherein the guide walls and/or the base body have/has predetermined separation points spaced proximodistally apart from one another and distributed over the guide length, each forming a mechanical weakening and being able to be severed in a defined manner.

4. The fixing device according to claim 1, wherein the guide walls are each formed by proximodistally spaced apart web elements with intermediate gaps.

5. The fixing device according to claim 4, wherein the intermediate gaps each form a length marking and/or a predetermined separation point.

6. The fixing device according to claim 1, wherein a proximodistal guide length measures between 10 mm and 60 mm.

7. The fixing device according to claim 1, wherein the fixing element and the guide recess are each arranged on an anterior top of the base body.

8. The fixing device according to claim 1, wherein the fixing element and the guide recess are each open anteriorly, as a result of which the catheter hub and the catheter tube portion are each insertable in a posterior direction into the fixing device.

9. The fixing device according to claim 1, wherein the fixing element and the guide recess are each open posteriorly, as a result of which the fixing device can be mounted in a posterior direction onto the catheter hub and the catheter tube portion.

10. The fixing device according to claim 9, wherein the posterior underside of the base body has a recess which extends proximodistally over an entire length of the base body and which divides the base body into a medial body half and a lateral body half and is formed in part by the guide recess, wherein the medial body half and the lateral body half are connected to each other at least by the fixing element.

11. The fixing device according to claim 1, further comprising a stop element arranged proximally on the base body relative to the fixing element and by which at least a proximal mobility of the catheter hub and/or of a fluid-carrying component of the catheter attached to the catheter hub is positively limited.

12. The fixing device according to claim 1, wherein the base body is made of a dimensionally stable plastics material.

13. The fixing device according to claim 1, wherein the base body has a proximodistal extent and a mediolateral extent, and wherein the base body is thin along a vertical axis and/or is designed as a proximodistally and mediolaterally planar plate relative to the proximodistal extent and the mediolateral extent.

14. The fixing device according to claim 1, further comprising a dimensionally flexible cushion body to which the base body is firmly applied, and of which the posterior underside is configured for placing on the skin.

15. The fixing device according to claim 1, further comprising an adhesive coating on an underside of the base body, the adhesive coating configured for releasably gluing the fixing device to the skin.
